(19) United States
(12) Patent Application Publication
(10) Pub. No.: US 2026/0200924 A1
(43) Pub. Date: Jul. 16, 2026

(54) CANNABINOID RECEPTOR 1 ANTAGONISTS AND USES

(71) Applicant: RESEARCH TRIANGLE INSTITUTE, Research Triangle Park, NC (US)

(72) Inventors: George S. Amato, Cary, NC (US); Rangan Maitra, Cary, NC (US)

(21) Appl. No.: 19/141,127

(22) PCT Filed: Dec. 20, 2023
(86) PCT No.: PCT/IB2023/063040

§ 371 Date: Jun. 19, 2025

Related U.S. Application Data
(60) Provisional application No. 63/433,793, filed on Dec. 20, 2022.

(51) Int. Cl. C07D 473/34; A61K 31/52; A61K 45/06; A61P 25/30

(52) U.S. Cl. CPC C07D 473/34; A61K 31/52; A61K 45/06; A61P 25/30

(57) ABSTRACT

The disclosure provides compounds capable of acting as antagonists at cannabinoid receptors according to the following formula: (I), wherein R is H or methyl; R′ is aryl or heteroaryl, optionally substituted with one or two substituents independently selected from the group consisting of F, Cl, methyl, CF₃, OCF₃, and OCHF₂, or a pharmaceutically acceptable ester, amide, salt, solvate, prodrug, or isomer thereof. Such compounds may be used to treat conditions for which the cannabinoid receptor system has been implicated, such as metabolic syndromes and substance use disorders.

### CROSS-REFERENCE TO RELATED APPLICATIONS

The present application claims priority to U.S. Provisional Patent Application No. 63/433,793, filed Dec. 20, 2022, which is incorporated herein by reference in its entirety.

### FEDERALLY SPONSORED RESEARCH OR DEVELOPMENT

This invention was made with United States government support under Grant Nos. R01 DK100414, R41 DK130765, and U18 DA052495 awarded by the National Institutes of Health. The government has certain rights in the invention.

### FIELD OF THE DISCLOSURE

The present application is directed to various compounds and methods of preparation of compounds that are capable of functioning as antagonists of cannabinoid (CB1) receptors. The application is also directed to pharmaceutical compositions containing one or more of these compounds, which may also contain one or more additional therapeutic agents. It is also directed to methods of treatment of various conditions that may be responsive to antagonism of the CB1 receptors, including, but not limited to, cannabis use disorder and metabolic syndrome.

### BACKGROUND

Cannabinoid receptors (CBRs) belong to the endocannabinoid (EC) system, which consists of receptors, transporters, endocannabinoids, and enzymes involved in synthesis and degradation of endocannabinoids. The EC system regulates many important physiological processes, and several components of the EC system are under evaluation as targets to treat a diverse array of indications including addiction, obesity, liver disease, diabetes, pain, and inflammation. To date, two different cannabinoid (“CB”) receptors have been identified (referred to as CB1 and CB2). CB1 and CB2 receptors fall within the class of G protein-coupled receptors (GPCRs), and primarily function to activate inhibitory G proteins (Gi/o). The CB1 receptor is prominently expressed in the central nervous system (CNS) and also in peripheral tissues. Accordingly, drugs targeting the CB1 receptors have been developed over the years to treat various disorders including addiction, obesity, and diabetes.

Antagonists generally can be classified as inverse agonists (which inactivate both intrinsic and extrinsic/ligand-stimulated receptor activities), neutral antagonists (which selectively block ligand-activated signaling) and partial inverse agonists (which can only block intrinsic signaling to a certain degree). The first drug selective for CB1 that was developed for medical use was rimonabant, a full inverse agonist. Rimonabant showed encouraging results in treating obesity and cannabis use disorder, and in smoking cessation. However, rimonabant was withdrawn from European markets and denied FDA approval in the United States due to CNS-related side effects including anxiety, depression, and suicidal ideation. The development of other related compounds (e.g., taranabant, otenabant, and ibipinabant) was discontinued based on these noted side effects. Accordingly, new CB1 antagonist compounds are needed for testing in humans.

### BRIEF SUMMARY

The present disclosure provides compounds useful as antagonists of the CB1 receptor, and methods of synthesis of such compounds. Further, some of these compounds are partial inverse agonists, which make them unique. It also provides pharmaceutical compositions containing the compounds, which may be useful in the treatment of various conditions and/or disorders. The disclosure further provides methods of treating such conditions and/or disorders, including but not limited to, metabolic syndrome, binge eating disorder, Prader Willi syndrome (PWS), and substance use disorders (SUD) including addiction to stimulants, depressants, cannabis, synthetic cannabinoids, alcohol consumption, nicotine consumption, acute cannabinoid induced toxicity, and cannabinoid hyperemesis syndrome. For example, in one aspect, the disclosure is directed to a method of treating a condition comprising administering to a subject in need of treatment of the condition a pharmaceutical composition comprising a therapeutically effective amount of a compound of the present disclosure or a pharmaceutically acceptable ester, amide, salt, solvate, prodrug, or isomer thereof.

The invention includes, without limitation, the following embodiments.

Embodiment 1: A compound according to the structure:

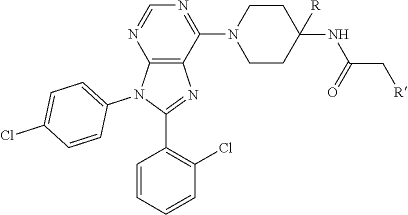

wherein: R is H or methyl; R′ is aryl or heteroaryl, optionally substituted with one or two substituents independently selected from the group consisting of F, Cl, methyl, CF₃, OCF₃, and OCHF₂, or a pharmaceutically acceptable ester, amide, salt, solvate, prodrug, or isomer thereof.

Embodiment 2: The compound of Embodiment 1, wherein R is H.

Embodiment 3: The compound of Embodiment 1, wherein R is methyl.

Embodiment 4: The compound of any of Embodiments 1-3, wherein R′ is aryl, optionally substituted with one or two substituents.

Embodiment 5: The compound of any of Embodiments 1-3, wherein R′ is heteroaryl, optionally substituted with one or two substituents.

Embodiment 6: The compound of any of Embodiments 1-5, wherein R′ is substituted with one or two substituents.

Embodiment 7: The compound of any of Embodiments 1-3, wherein R′ is phenyl substituted with one or two F substituents.

Embodiment 8: The compound of any of Embodiments 1-3, wherein R′ is selected from the group consisting of:

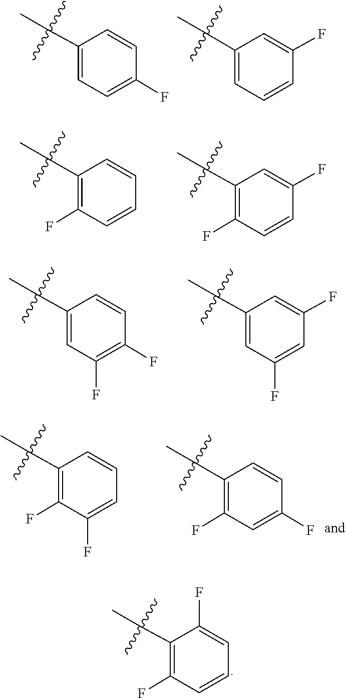

Embodiment 9: The compound of any of Embodiments 1-8, in the form of a pharmaceutically acceptable salt.

Embodiment 10: A pharmaceutical composition, comprising the compound of any of Embodiments 1-9 and one or more pharmaceutically acceptable carriers.

Embodiment 11: The pharmaceutical composition of Embodiment 10, further comprising one or more additional active agents.

Embodiment 12: A method for treating or delaying the progression of disorders that are alleviated by antagonism of the CB1 receptor, the method comprising administering a compound according to any of Embodiments 1-9 or a pharmaceutical composition according to either of Embodiments 10 or 11.

Embodiment 13: The method of Embodiment 12, wherein the disorder is selected from the group consisting of metabolic syndrome, binge eating disorder, Preder Willi syndrome (PWS), and substance use disorders.

Embodiment 14: The method of Embodiment 13, wherein the substance use disorder is selected from the group consisting of addiction to stimulants, depressants, or cannabis, alcohol consumption, nicotine consumption, acute cannabinoid-induced toxicity, and cannabinoid hyperemesis syndrome.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawings, which are not necessarily drawn to scale. The drawings are examples only and should not be construed as limiting the disclosure.

FIG. 1A is a plot of data obtained from a high-resolution functional fluorescence calcium mobilization assay conducted for a compound according to one, non-limiting embodiment of the disclosure;

FIG. 1B is a plot of data evaluating % normalized baseline response of rimonabant and a compound according to one, non-limiting embodiment of the disclosure;

FIG. 2 is a plot of data obtained from a study of brain penetration and plasma clearance for a compound according to one, non-limiting embodiment of the disclosure;

FIG. 3 is a plot of data obtained from a THC drug discrimination assay in mice for a compound according to one, non-limiting embodiment of the disclosure;

FIGS. 4A, 4B, and 4C are plots of data obtained from reversal of diet-induced obesity (DIO) and metabolic syndrome in mice for a compound according to one, non-limiting embodiment of the disclosure;

FIGS. 4D, 4E, and 4F are plots of data demonstrating reduction of circulating transaminases and decreased hepatic cholesterol content in mice following treatment with a compound according to one, non-limiting embodiment of the disclosure;

FIG. 4G is a plot of data demonstrating reduction of hepatic steatosis in mice following treatment with a compound according to one, non-limiting embodiment of the disclosure;

FIGS. 4H, 4I, and 4J are representative images of the reduced hepatic steatosis plotted in FIG. 4G;

FIG. 5A is a representative image of target specificity of a non-limiting compound of one, non-limiting embodiment of the disclosure for the CB1 receptor in mice; and

FIG. 5B is a plot showing blockade of signal upon pre-treatment in mice with a compound according to one, non-limiting embodiment of the disclosure.

### DETAILED DESCRIPTION

The present invention now will be described more fully hereinafter. However, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The present disclosure provides compounds that exhibit activity at the cannabinoid (e.g., CB1) receptor, as well as methods of preparation and pharmaceutical compositions thereof. It also provides methods for using such compounds to treat a variety of disorders that may be responsive to the antagonism of CB1 receptors. In particular, the compositions and methods can be used in the treatment of obesity, metabolic syndrome, and cannabis use disorder (CUD). Treatment can comprise the use of a compound of the present disclosure as a single active agent. In other embodiments, treatment can comprise the use of a compound of the present disclosure in combination with one or more further active agents. The specific pharmaceutical composition (or compositions) used in the disclosure, and the methods of treatment provided by the disclosure, are further described below.

### Definitions

The term “alkyl” as used herein means saturated straight, branched, or cyclic hydrocarbon groups (i.e., cycloalkyl groups). In particular embodiments, alkyl refers to groups comprising 1 to 10 carbon atoms (“C1-10 alkyl”). In further embodiments, alkyl refers to groups comprising 1 to 8 carbon atoms (“C1-8 alkyl”), 1 to 6 carbon atoms (“C1-6 alkyl”), or 1 to 4 carbon atoms (“C1-4 alkyl”). In other embodiments, alkyl refers to groups comprising 3-10 carbon atoms (“C3-10 alkyl”), 3-8 carbon atoms (“C3-8 alkyl”), or 3-6 carbon atoms (“C3-6 alkyl”). In specific embodiments, alkyl refers to methyl, trifluoromethyl, ethyl, propyl, isopropyl, cyclopropyl, butyl, isobutyl, sec-butyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl.

The term “heteroalkyl” as used herein means an alkyl group, having at least one atom within the chain which is not carbon. Preferred heteroatoms include sulfur, oxygen, and nitrogen.

“Optionally substituted” in reference to a substituent group refers to substituent groups optionally substituted with one or more moieties selected from the group consisting of halo (e.g., Cl, F, Br, and I); halogenated alkyl (e.g., CF₂, 2-Br-ethyl, CH₂F, CH₂Cl, CH₂CF₃, or CF₂CF₃); hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate.

The term “alkenyl” as used herein means alkyl moieties wherein at least one saturated C—C bond is replaced by a double bond. In particular embodiments, alkenyl refers to groups comprising 2 to 10 carbon atoms (“C2-10 alkenyl”). In further embodiments, alkenyl refers to groups comprising 2 to 8 carbon atoms (“C2-8 alkenyl”), 2 to 6 carbon atoms (“C2-6 alkenyl”), or 2 to 4 carbon atoms (“C2-4 alkenyl”). In specific embodiments, alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl.

The term “alkynyl” as used herein means alkyl moieties wherein at least one saturated C—C bond is replaced by a triple bond. In particular embodiments, alkynyl refers to groups comprising 2 to 10 carbon atoms (“C2-10 alkynyl”). In further embodiments, alkynyl refers to groups comprising 2 to 8 carbon atoms (“C2-8 alkynyl”), 2 to 6 carbon atoms (“C2-6 alkynyl”), or 2 to 4 carbon atoms (“C2-4 alkynyl”). In specific embodiments, alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkoxy” as used herein means straight or branched chain alkyl groups linked by an oxygen atom (i.e., —O-alkyl), wherein alkyl is as described above. In particular embodiments, alkoxy refers to oxygen-linked groups comprising 1 to 10 carbon atoms (“C1-10 alkoxy”). In further embodiments, alkoxy refers to oxygen-linked groups comprising 1 to 8 carbon atoms (“C1-8 alkoxy”), 1 to 6 carbon atoms (“C1-6 alkoxy”), 1 to 4 carbon atoms (“C1-4 alkoxy”) or 1 to 3 carbon atoms (“C1-3 alkoxy”).

The term “halo” or “halogen” as used herein means fluorine, chlorine, bromine, or iodine.

The term “alkylthio” as used herein means a thio group with one or more alkyl substituents, where alkyl is defined as above.

The terms “aralkyl” and “arylalkyl” as used herein mean an aryl group as defined below linked to the molecule through an alkyl group as defined above.

The terms “alkaryl” and “alkylaryl” as used herein means an alkyl group as defined above linked to the molecule through an aryl group as defined below.

The term “alkylarylalkyl” as used herein means an alkyl group as defined above linked to the molecule through an arylalkyl group as defined above.

The term “amino” as used herein means a moiety represented by the structure NR₂, and includes primary amines, and secondary and tertiary amines substituted by alkyl or aryl (i.e., alkylamino or arylamino, respectively). Thus, R2 may represent two hydrogen atoms, two alkyl moieties, two aryl moieties, one aryl moiety and one alkyl moiety, one hydrogen atom and one alkyl moiety, or one hydrogen atom and one aryl moiety.

The term “cycloalkyl” means a non-aromatic, monocyclic or polycyclic ring comprising carbon and hydrogen atoms.

The term “heterocycloalkyl” means a cycloalkyl group, having at least one atom within the ring which is not carbon. Preferred heteroatoms include sulfur, oxygen, and nitrogen.

The term “aryl” as used herein means a stable monocyclic, bicyclic, or tricyclic carbon ring of up to 8 members in each ring, wherein at least one ring is aromatic as defined by the Hückel 4n+2 rule. Exemplary aryl groups according to the disclosure include phenyl, naphthyl, tetrahydronaphthyl, and biphenyl.

The term “heteroaryl” as used herein means an aryl group, having at least one atom within the ring which is not carbon. Preferred heteroatoms include sulfur, oxygen, and nitrogen.

The term “derivative” as used herein means a compound that is formed from a similar, beginning compound by attaching another molecule or atom to the beginning compound. Further, derivatives, according to the disclosure, encompass one or more compounds formed from a precursor compound through addition of one or more atoms or molecules or through combining two or more precursor compounds.

The term “prodrug” as used herein means any compound which, when administered to a mammal, is converted in whole or in part to a compound of the disclosure.

The term “active metabolite” as used herein means a physiologically active compound which results from the metabolism of a compound of the present disclosure, or a prodrug thereof, when such compound or prodrug is administered to a mammal.

The terms “therapeutically effective amount” or “therapeutically effective dose” as used herein are interchangeable and mean a concentration of a compound according to the disclosure, or a biologically active variant thereof, sufficient to elicit the desired therapeutic effect according to the methods of treatment described herein.

The term “pharmaceutically acceptable carrier” as used herein means a carrier that is conventionally used in the art to facilitate the storage, administration, and/or the healing effect of a biologically active agent.

The term “intermittent administration” as used herein means administration of a therapeutically effective dose of a composition according to the present disclosure, followed by a time period of discontinuance, which is then followed by another administration of a therapeutically effective dose, and so forth.

### Active Agents

The present disclosure provides compounds, methods of preparation of the compounds, pharmaceutical compositions, and methods of treatment of various conditions using such compounds and pharmaceutical compositions.

In some embodiments, compounds according to the following general structure are provided:

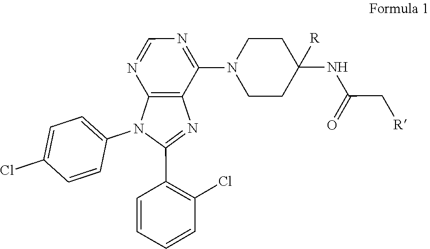

wherein:
   R is H or methyl;
   R′ is aryl or heteroaryl, optionally substituted with one or two substituents independently selected from the group consisting of F, Cl, Me, CF₃, OCF₃, and OCHF₂,
or a pharmaceutically acceptable ester, amide, salt, solvate, prodrug, or isomer thereof.

In some embodiments, R is H. In some embodiments, R is Me.

In some embodiments, R′ is optionally substituted aryl. For example, R′ can be optionally substituted phenyl. In some embodiments, R′ is optionally substituted heteroaryl. For example, R′ can be optionally substituted pyridine. In some embodiments, R′ is optionally substituted oxadiazole. In some embodiments, R′ is optionally substituted oxazole. In some embodiments, R′ is optionally substituted thiazole. In some embodiments, R′ is optionally substituted indole. In further embodiments, R′ is optionally substituted pyrimidine, pyrazine, or pyridazine.

In some embodiments, R′ is unsubstituted aryl or heteroaryl. In some embodiments, R′ is aryl or heteroaryl with one substituent selected from F, Cl, Me, CF₃, OCF₃, and OCHF₂. In some embodiments, R′ is aryl or heteroaryl with two substituents, independently selected from F, Cl, Me, CF₃, OCF₃, and OCHF₂.

In some embodiments, R′ is aryl or heteroaryl substituted with one F substituent. In some preferred embodiments, R′ is aryl or heteroaryl substituted with two F substituents. In some embodiments, R′ is phenyl substituted with one F substituent. In some embodiments, R′ is phenyl substituted with two F substituents. For example, in some embodiments, R′ is selected from the following:

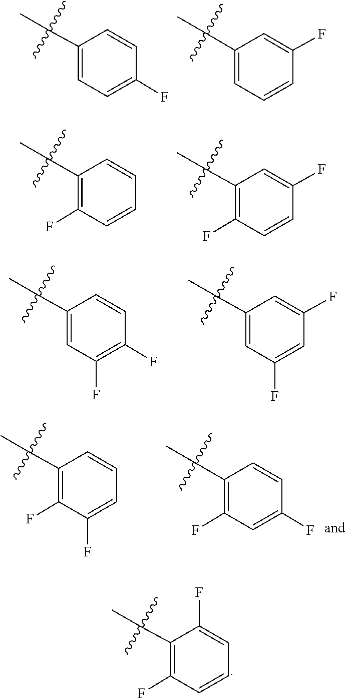

The referenced F substituent(s) in these embodiments can, in some embodiments, be the only substituent(s) present on the aryl or heteroaryl. In other embodiments, the aryl or heteroaryl can comprise one F substituent and can further comprise one or more additional substituents selected from Cl, Me, CF₃, OCF₃, and OCHF₂.

Certain compounds according to the present disclosure exhibit partial blockade of CB1 basal signaling (e.g., about 30%). Advantageously, although exhibiting only partial blockade of CB1 basal signaling, compounds as disclosed herein nonetheless exhibit excellent binding affinity and selectivity toward CB1 receptors (versus CB2 receptors), as demonstrated, e.g., in the data presented herein below.

The compounds disclosed herein as active agents may contain chiral centers, which may be either of the (R) or (S) configuration, or may comprise a mixture thereof. Accordingly, the present disclosure also includes stereoisomers of the compounds described herein, where applicable, either individually or admixed in any proportions. Stereoisomers may include, but are not limited to, enantiomers, diastereomers, racemic mixtures, and combinations thereof. Such stereoisomers can be prepared and separated using conventional techniques, either by reacting enantiomeric starting materials, or by separating isomers of compounds of the present disclosure. Isomers may include geometric isomers. Examples of geometric isomers include, but are not limited to, cis isomers or trans isomers across a double bond. Other isomers are contemplated among the compounds of the present disclosure. The isomers may be used either in pure form or in admixture with other isomers of the compounds described herein.

In some embodiments, the compounds of Formula 1 are racemic. In some embodiments, compounds with one or more chiral centers are provided. While racemic mixtures of compounds of the disclosure can be active, selective, and bioavailable, isolated isomers may be of interest as well. The compounds of the present disclosure optionally may be provided in a composition that is enantiomerically enriched, such as a mixture of enantiomers in which one enantiomer is present in excess, in particular to the extent of 95% or more, or 98% or more, including 100%.

Various methods are known in the art for preparing optically active forms and determining activity. Such methods include standard tests described herein other similar tests which are well known in the art. Examples of methods that can be used to obtain optical isomers of the compounds according to the present disclosure include the following:
i) physical separation of crystals whereby macroscopic crystals of the individual enantiomers are manually separated. This technique may particularly be used when crystals of the separate enantiomers exist (i.e., the material is a conglomerate), and the crystals are visually distinct;
ii) simultaneous crystallization whereby the individual enantiomers are separately crystallized from a solution of the racemate, possible only if the latter is a conglomerate in the solid state;
iii) enzymatic resolutions whereby partial or complete separation of a racemate by virtue of differing rates of reaction for the enantiomers with an enzyme;
iv) enzymatic asymmetric synthesis, a synthetic technique whereby at least one step of the synthesis uses an enzymatic reaction to obtain an enantiomerically pure or enriched synthetic precursor of the desired enantiomer;
v) chemical asymmetric synthesis whereby the desired enantiomer is synthesized from an achiral precursor under conditions that produce asymmetry (i.e., chirality) in the product, which may be achieved using chiral catalysts or chiral auxiliaries;
vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors whereby the desired enantiomer is obtained from non-chiral starting materials and where the stereochemical integrity is not or is only minimally compromised over the course of the synthesis;
x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography whereby the racemate is volatilized and enantiomers are separated by virtue of their differing interactions in the gaseous mobile phase with a column containing a fixed non-racemic chiral adsorbent phase;
xii) extraction with chiral solvents whereby the enantiomers are separated by virtue of preferential dissolution of one enantiomer into a particular chiral solvent; and
xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The terms (R) and(S) as used herein mean that the composition contains a greater proportion of the named isomer of the compound in relation to other isomers. In a preferred embodiment these terms indicate that the composition contains at least 90% by weight of the named isomer and 10% by weight or less of the one or more other isomers; or more preferably about 95% by weight of the named isomer and 5% or less of the one or more other isomers. These percentages are based on the total amount of the compound of the present disclosure present in the composition.

The compounds of the present disclosure may be utilized per se or in the form of a pharmaceutically acceptable ester, amide, salt, solvate, prodrug, or isomer. For example, the compound may be provided as a pharmaceutically acceptable salt. If used, a salt of the drug compound should be both pharmacologically and pharmaceutically acceptable, but non-pharmaceutically acceptable salts may conveniently be used to prepare the free active compound or pharmaceutically acceptable salts thereof and are not excluded from the scope of this disclosure. Such pharmacologically and pharmaceutically acceptable salts can be prepared by reaction of the drug with an organic or inorganic acid, using standard methods detailed in the literature. Examples of pharmaceutically acceptable salts of the compounds useful according to the disclosure include acid addition salts. Salts of non-pharmaceutically acceptable acids, however, may be useful, for example, in the preparation and purification of the compounds. Suitable acid addition salts according to the present disclosure include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzenesulfonic, and isethionic acids. Other useful acid addition salts include propionic acid, glycolic acid, oxalic acid, malic acid, malonic acid, benzoic acid, cinnamic acid, mandelic acid, salicylic acid, and the like. Particular example of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, phthalates, methoxyenzoates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

An acid addition salt may be reconverted to the free base by treatment with a suitable base. Preparation of basic salts of acid moieties which may be present on a compound useful according to the present disclosure may be prepared in a similar manner using a pharmaceutically acceptable base, such as sodium hydroxide, potassium hydroxide, ammonium hydroxide, calcium hydroxide, triethylamine, or the like.

Esters of the active agent compounds according to the present disclosure may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Moreover, esters, urease, sulfonamides, and amides of compounds of the disclosure can be made by reaction with a carbonylating agent (e.g., ethyl formate, acetic anhydride, methoxyacetyl chloride, benzoyl chloride, methyl isocyanate, ethyl chloroformate) or methanesulfonyl chloride and a suitable base (e.g., 4-dimethylaminopyridine, pyridine, triethylamine, potassium carbonate) in a suitable organic solvent (e.g., tetrahydrofuran, acetone, methanol, pyridine, N,N-dimethylformamide) at a temperature of 0° C. to 60° C. Prodrugs are typically prepared by covalent attachment of a moiety, which results in a compound that is therapeutically inactive until modified by an individual's metabolic system. Examples of pharmaceutically acceptable solvates include, but are not limited to, compounds according to the disclosure in combination with water, isopropanol, ethanol, methanol, DMSO, ethyl acetate, acetic acid, or ethanolamine.

In the case of solid compositions, it is understood that the compounds used in the methods of the present disclosure may exist in different forms. For example, the compounds may exist in stable and metastable crystalline forms and isotropic and amorphous forms, all of which are intended to be within the scope of the present disclosure.

If a compound useful as an active agent according to the disclosure is a base, the desired salt may be prepared by any suitable method known to the art, including treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, pyranosidyl acids such as glucuronic acid and galacturonic acid, alpha-hydroxy acids such as citric acid and tartaric acid, amino acids such as aspartic acid and glutamic acid, aromatic acids such as benzoic acid and cinnamic acid, sulfonic acids such a p-toluenesulfonic acid or ethanesulfonic acid, or the like.

If a compound described herein as an active agent is an acid, the desired salt may be prepared by any suitable method known to the art, including treatment of the free acid with an inorganic or organic base, such as an amine (primary, secondary, or tertiary), an alkali metal or alkaline earth metal hydroxide or the like. Illustrative examples of suitable salts include organic salts derived from amino acids such as glycine and arginine, ammonia, primary, secondary, and tertiary amines, and cyclic amines such as piperidine, morpholine and piperazine, and inorganic salts derived from sodium, calcium, potassium, magnesium, manganese, iron, copper, zinc, aluminum, and lithium.

The present disclosure further includes prodrugs and active metabolites of the active agent compounds described herein. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound.

A number of prodrug ligands are known. In general, alkylation, acylation, or other lipophilic modification of one or more heteroatoms of the compound, such as a free amine or carboxylic acid residue, reduces polarity and allows passage into cells. Examples of substituent groups that can replace one or more hydrogen atoms on the compounds of the present disclosure include, but are not limited to, the following: aryl; steroids; carbohydrates (including sugars); 1,2-diacylglycerol; alcohols; acyl (including lower acyl); alkyl (including lower alkyl); sulfonate ester (including alkyl or arylalkyl sulfonyl, such as methanesulfonyl and benzyl, wherein the phenyl group is optionally substituted with one or more substituents as provided in the definition of an aryl given herein); optionally substituted arylsulfonyl; lipids (including phospholipids); phosphotidylcholine; phosphocholine; amino acid residues or derivatives; amino acid acyl residues or derivatives; peptides; cholesterols; or other pharmaceutically acceptable leaving groups which, when administered in vivo, provide the free moiety, e.g., amine and/or carboxylic acid moiety. Any of these can be used in combination with the disclosed active agents to achieve a desired effect.

Certain preferred compounds of the present disclosure include the following:

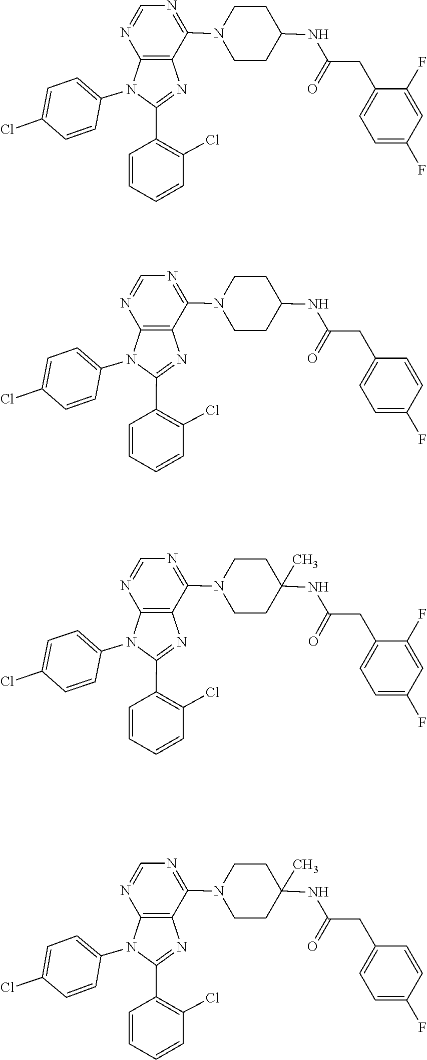 [SMILES: O=C(Cc1ccc(F)cc1F)NC1CCN(c2ncnc3c2nc(-c2ccccc2Cl)n3-c2ccc(Cl)cc2)CC1 ; O=C(Cc1ccc(F)cc1)NC1CCN(c2ncnc3c2nc(-c2ccccc2Cl)n3-c2ccc(Cl)cc2)CC1 ; CC1(NC(=O)Cc2ccc(F)cc2F)CCN(c2ncnc3c2nc(-c2ccccc2Cl)n3-c2ccc(Cl)cc2)CC1 ; CC1(NC(=O)Cc2ccc(F)cc2)CCN(c2ncnc3c2nc(-c2ccccc2Cl)n3-c2ccc(Cl)cc2)CC1]

The compounds of the present disclosure may function as partial inverse agonists at the CB1 receptor. These compounds preserve basal/intrinsic activity of the CB1 receptor by at least 50% or more compared to the full inverse agonist rimonabant. In preferred embodiments, the compounds of the present disclosure are selective for the CB1 receptor (e.g., over the CB2 receptor).

### Methods of Preparation

The present disclosure also encompasses methods of preparing compounds with structures encompassed by Formula 1. One of skill in the art would be able to adapt these methods as required to accommodate various functional groups that may affect the chemistry of the synthesis.

Scheme 1 shows a general synthesis used for the production of various compounds according to Formula 1. Briefly, intermediate A is prepared from commercially available 5-amino-4,6-dichloropyrimidine using a two-step procedure (a), chloro displacement with 4-chloroaniline followed by cyclization with 2-chlorobenzaldehyde. Conversion of A to B is achieved in a two-step process (b), comprising a chloro displacement with a Boc-protected 4-aminopiperidine followed by hydrolysis of the Boc group. From B, final products C are prepared with a standard amide-forming reaction (c), for example using HBTU as the coupling agent.

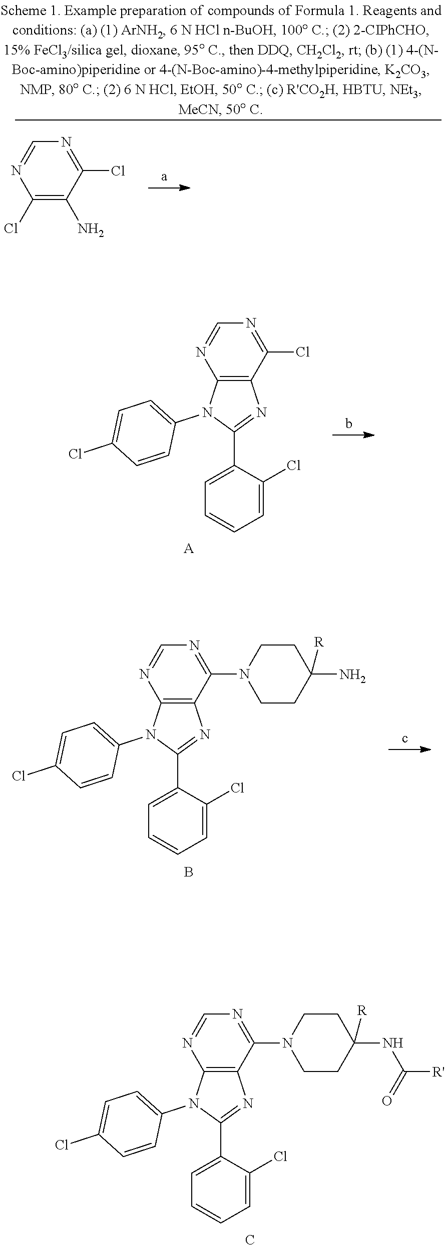

### Compositions

While it is possible for the compounds of the present disclosure to be administered in the raw chemical form, it is preferred for the compounds to be delivered as a pharmaceutical formulation. Accordingly, the present disclosure provides pharmaceutical compositions comprising at least one compound capable of functioning as an antagonist of the CB1 receptor. As such, the formulations of the present disclosure comprise a compound of Formula 1, as described above, or a pharmaceutically acceptable ester, amide, salt, or solvate thereof, together with one or more pharmaceutically acceptable carriers therefore, and optionally, other therapeutic ingredients.

By “pharmaceutically acceptable carrier” is intended a carrier that is conventionally used in the art to facilitate the storage, administration, and/or the healing effect of the agent. The carrier(s) must be pharmaceutically acceptable in the sense of being compatible with the other ingredients of the formulation and not unduly deleterious to the recipient thereof. A carrier may also reduce any undesirable side effects of the agent. Such carriers are known in the art. See, Wang et al. (1980) J. Parent. Drug Assn. 34 (6): 452-462, herein incorporated by reference in its entirety.

Adjuvants or accessory ingredients for use in the formulations of the present disclosure can include any pharmaceutical ingredient commonly deemed acceptable in the art, such as binders, fillers, lubricants, disintegrants, diluents, surfactants, stabilizers, preservatives, flavoring and coloring agents, and the like. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations).

Exemplary pharmaceutical excipients and/or additives suitable for use in the compositions according to the disclosure are listed in Remington: The Science & Practice of Pharmacy,” 21^{st} ed. Lippincott Williams & Wilkins (2006); in the Physician's Desk Reference, 64^{th} ed., Thomson PDR (2010); and in Handbook of Pharmaceutical Excipients, 6^{th} ed., Eds. Raymond C. Rowe et al., Pharmaceutical Press (2009), which are incorporated herein by reference.

Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluloses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present disclosure may be anionic, cationic, amphoteric, or nonionic surface-active agents. Stabilizers may be included in the formulations to inhibit or lessen reactions leading to decomposition of the active agent, such as oxidative reactions.

Formulations of the present disclosure may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pennsylvania, 1990), herein incorporated by reference in its entirety.

Pharmaceutical formulations according to the present disclosure are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent, such as the compounds of Formula 1 according to the present disclosure (or a pharmaceutically acceptable ester, amide, salt, or solvate thereof) with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

Pharmaceutical formulations according to the present disclosure suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of the above dosage forms are generally known in the art, and any such method would be suitable for the preparation of the respective dosage forms for use in delivery of the compounds according to the present disclosure.

A tablet containing a compound according to the present disclosure may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present disclosure may also be sustained release (i.e., releasing the active agent over a prolonged period of time), and may or may not also be delayed release. Sustained release formulations are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Formulations for parenteral administration include aqueous and non-aqueous sterile injection solutions, which may further contain additional agents, such as antioxidants, buffers, bacteriostats, and solutes, which render the formulations isotonic with the blood of the intended recipient. The formulations may include aqueous and non-aqueous sterile suspensions, which contain suspending agents and thickening agents. Such formulations for parenteral administration may be presented in unit-dose or multi-dose containers, such as, for example, sealed ampoules and vials, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present disclosure may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Formulations for rectal delivery of the compounds of the present disclosure include rectal suppositories, creams, ointments, and liquids. Suppositories may be presented as the active agent in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

The compounds of Formula 1 above may be formulated in compositions including those suitable for oral, buccal, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing a compound of Formula 1 into association with a carrier that constitutes one or more accessory ingredients. In general, the compositions are prepared by bringing a compound of the disclosure into association with a liquid carrier to form a solution or a suspension, or alternatively, bringing a compound of the disclosure into association with formulation components suitable for forming a solid, optionally a particulate product, and then, if warranted, shaping the product into a desired delivery form. Solid formulations of the disclosed compounds, when particulate, will typically comprise particles with sizes ranging from about 1 nanometer to about 500 microns. In general, for solid formulations intended for intravenous administration, particles will typically range from about 1 nm to about 10 microns in diameter.

The amount of the compound of Formula 1 in the formulation will vary depending on the specific compound selected, dosage form, target patient population, and other considerations, and will be readily determined by one skilled in the art. The amount of the compound of Formula 1 in the formulation will be that amount necessary to deliver a therapeutically effective amount of the compound to a patient in need thereof to achieve at least one of the therapeutic effects associated with the compounds of the present disclosure. In practice, this will vary widely depending upon the particular compound, its activity, the severity of the condition to be treated, the patient population, the stability of the formulation, and the like. Compositions will generally contain anywhere from about 1% by weight to about 99% by weight of a compound of the disclosure, typically from about 5% to about 70% by weight, and more typically from about 10% to about 50% by weight, and will also depend upon the relative amounts of excipients/additives contained in the composition.

### Combinations

In specific embodiments, active agents used in combination with compounds of the present disclosure comprise one or more compounds generally recognized as useful for treating the conditions discussed herein. In one embodiment, the use of two or more drugs, which may be of different therapeutic classes, may enhance efficacy and/or reduce adverse effects associated with one or more of the drugs.

For example, in certain embodiments, the present disclosure relates to the treatment of obesity. Accordingly, in one embodiment, a compound of Formula 1 is combined with one or more known anti-obesity drugs for the treatment of obesity. Common therapeutic classes of obesity drugs include those that decrease food intake by either reducing appetite or increasing satiety, those that decrease nutrient absorption, and those that increase energy expenditure. Examples of known anti-obesity drugs include, but are not limited to: phentermine, which is an appetite suppressant; topiramate, which is an depressant/epilepsy drug that has been shown to interfere with binge eating and may result in decreased weight and decreased blood pressure; Orlistat (Xenical, Alli®), which reduces intestinal fat absorption by inhibiting pancreatic lipase; Sibutramine (Reductil or Meridia), which is an anorectic or appetite suppressant; diethylpropion (diethylcathinone/amfepramone, also sold as Anorex,® Tenuate,® and Tepanil®), which is a stimulant marketed as an appetite suppressant (which functions as a prodrug for ethcathinone); Mazindol (Mazanor, Sanorex), which is a tetracyclic stimulant drug used for short-term treatment of obesity; metformin (glucophage) in people with diabetes mellitus type 2; and Exenatide (Byetta), Pramlintide (Symlin), and Rybelsus, which delay gastric emptying and promote a feeling of satiety. Combination therapies using a compound of the present disclosure 1 along with an antidepressant such as a selective serotonin reuptake inhibitor (SSRI) or serotonergic ligand such as trazodone, citalopram, dapoxetine, escitalopram, fluoxetine, fluvoxamine, paroxetine, or sertraline, may be advantageous, e.g., by functioning to further reduce obesity while promoting emotional welfare. Other over-the-counter weight loss products including herbal remedies, laxatives, diet pills, diuretic drugs, and/or pyruvate may also be combined with the compounds disclosed herein. The compounds disclosed herein may also be used in combination with non drug-based therapy, including caloric restriction, weight-loss surgery, exercise, and behavioral therapy.

Combinations of compounds of the present disclosure with other therapeutic agents are also included in the present disclosure, wherein the condition to be treated is any condition that is responsive to the antagonism of the CB1 receptor.

For example, diabetes may be treated with compounds of the present disclosure, and thus, in one embodiment, a compound of Formula 1 is combined with one or more known drugs for the treatment of diabetes. In certain embodiments, diabetes is treated with compounds of the present disclosure in combination with insulin. Diabetes medications generally fall within six classes of drugs that work in different ways to lower blood glucose levels. Specifically, these medications include sulfonylureas, which stimulate the beta cells of the pancreas to release more insulin (e.g., chlorpropamide (Diabinese), glipizide (Glucotrol and Glucotrol XL), glyburide (Micronase, Glynase, and Diabeta, and glimepiride (Amaryl)); meglitinides, which stimulate the beta cells to release insulin (e.g., repaglinide (Prandin) and nateglinide (Starlix)); biguanides, which lower blood glucose levels primarily by reducing the glucose produced by the liver (e.g., metformin (Glucophage)); thiazolidinediones, which help insulin to work better in the muscle and fat, and also reduce glucose production in the liver (e.g., rosiglitazone (Avandia) and pioglitazone (ACTOS)); alpha-glucosidase inhibitors, which help lower blood glucose levels by blocking the breakdown of starches in the intestine and may slow the breakdown of some sugars (e.g., acarbose (Precose) and meglitol (Glyset)); and DPP-4 inhibitors, which prevent the breakdown of GLP-1, which is a naturally occurring compound in the body that reduces blood glucose levels (e.g., sitagliptin (Januvia) and saxagliptin (Onglyza)).

Dyslipidemia may also be treated using compounds with the present disclosure. Thus, in one embodiment, a compound of Formula 1 is combined with one or more known drugs for the treatment of dyslipidemia. Medications for dyslipidemia typically fall into four classes of compounds capable of lowering lipid levels. These classes include statins, which are 3-hydroxy-3-methylglutaryl coenzyme A (HMG-COA) reductase inhibitors (e.g., rosuvastatin, lovastatin, atorvastatin, pravastatin, fluvastatin, pitavastatin, and simvastatin); fibrates, which reduce triglyceride and very low-density lipoprotein production in the liver (e.g., gemfibrozil, clofibrate, and fenofibrate); niacin (also known as nicotinic acid or Vitamin B3), which lowers total cholesterol and triglycerides and may also increase high-density lipoprotein cholesterol; and bile acid sequestering resins, which bind bile acids in the small intestine and prevent their return to the liver (e.g., cholestipol and cholestyramine).

Various liver diseases may be treated using compounds of the present disclosure. Accordingly, in one embodiment, a compound of Formula 1 is combined with one or more known drugs for the treatment of various types of liver disease. For example, exemplary medications used to treat fatty liver disease or nonalcoholic steatohepatitis include Actos, Avandia, Xenical, Actigall, Urso, Urso Forte, Orlostat, and Cystadane.

Further, in one embodiment, a compound of Formula 1 is combined with one or more known drugs for the treatment of pain and/or inflammation. Many such drugs are well known, and include, for example, acetaminophen (e.g., Tylenol and aspirin-free Excedrin); nonsteroidal anti-inflammatory drugs (NSAIDS, e.g., aspirin, Motrin, and Aleve); topical corticosteroids (e.g., Cortaid and Cortizone); corticosteroids (e.g., Deltasone, Hydeltrasol, and Solu-Medrol); opiods (e.g., morphine, fentanyl, oxycodone, and codeine); antidepressants (e.g., selective serotonin reuptake inhibitors (SSRIs) such as Celexa, Prozac, Paxil, and Zoloft; tricyclic antidepressants such as Elavil, Norpramin, Sinequan, Tofranil, and Pamelor; and selective serotonin and norepinephrine reuptake inhibitors (SSNRIs) such as Effexor and Cymbalta); and anticonvulsants (e.g., Tegretol, Neurontin, and Lyrica).

The compound of Formula 1 and the one or more other therapeutic agents may be contained within a single composition or alternatively may be administered concurrently or sequentially (consecutively) in any order. For sequential administration, each of the compound of Formula I and the one or more other therapeutic agents can be formulated in its own pharmaceutical composition, each of which is to be administered sequentially, in any order. Alternatively, the compound of Formula 1 and the one or more other therapeutic agents can be formulated together. The compositions may be formulated for oral, systemic, topical, intravenous, intraparenteral, intravaginal, intraocular, transbuccal, transmucosal, or transdermal administration.

### Methods of Use

In a further embodiment, the present disclosure provides a method for treating or delaying the progression of disorders that are alleviated by antagonizing the CB1 receptors in a patient, the method comprising administering a therapeutically effective amount of at least one compound of Formula 1 to the patient. Disorders that may be effectively alleviated to some degree by the compounds of the present disclosure include, but are not limited to, metabolic syndrome related complications, binge eating disorder, Prader Willi syndrome (PWS), lung fibrosis, and gastrointestinal diseases (including inflammatory bowel diseases and irritable bowel syndrome). The method of treatment generally includes administering a therapeutically effective amount of a compound of Formula 1, optionally in a pharmaceutical composition including one or more pharmaceutically acceptable carriers. The therapeutically effective amount is preferably sufficient to antagonize the CB1 receptor. The therapeutically effective amount is further preferably sufficient to cause some relief to the patient in the symptoms of the disorder/disease for which the patient is being treated.

In one embodiment, the present disclosure relates to the field of treating obesity in animals, particularly humans and other mammals, and associated effects of these conditions. It also may relate to the treatment of other conditions that may benefit from the antagonism of CB1 receptors, such as diabetes, liver diseases, liver fibrosis, dyslipidemia, pain/inflammation, metabolic disorder, kidney disease, cholestatic injury, kidney disease, lung fibrosis, addiction to cannabinergic agents, alcoholism, addiction to nicotine or stimulants, and gastrointestinal diseases (including inflammatory bowel diseases and irritable bowel syndrome).

In another embodiment, a method of treating substance use disorders (e.g., addiction) is provided. Such addiction may be, for example, to stimulants, depressants, and/or cannabis (including synthetic cannabis and cannabinoids). The substance use disorder can, in various embodiments, be cannabis use disorder (CUD), alcohol consumption, nicotine consumption, acute cannabinoid-induced toxicity, or cannabinoid hyperemesis syndrome. In such methods, a therapeutically effective amount of a compound of the present disclosure to treat a patient with CUD may be that amount capable of antagonizing the CB1 receptor. Such compound may cause the patient to experience decreased substance (e.g., stimulant, depressant, or cannabis) use.

In one embodiment, a method of treating obesity is provided. Obesity has its common meaning, e.g., the medical condition that exists when an individual has accumulated excess body fat, which may lead to a variety of related health problems, and which is characterized by a body mass index (BMI) of 30 kg/m² or more. Pre-obesity, also known as overweight, refers to the condition wherein an individual's BMI is between 25 kg/m² and 30 kg/m². In such methods, a therapeutically effective amount of a compound of the present disclosure to treat a patient with pre-obesity or obesity may be that amount capable of antagonizing the CB1 receptor. Such compound may cause the patient to experience decreased appetite and/or may create a sensation of fullness. The method of treating obesity may be used to attain or maintain a patient's weight loss.

In another embodiment, a method of treating liver disease is provided. The liver disease may be, for example, fatty liver disease or nonalcoholic steatohepatitis (e.g., obesity-related steatosis). For example, compounds of the present disclosure can, in some embodiments, be used to slow the development of fatty liver disease (alcoholic or non-alcoholic fatty liver) and, in some cases, prevent the progression of fatty liver to more severe forms of liver disease and/or liver fibrosis. In some embodiments, compounds of the present disclosure may function to provide hepatoprotective activity. In some embodiments, the compounds may be capable of modulating lipid levels, reducing cholesterol, free fatty acids, and/or triglycerides.

In one embodiment, a method of treating diabetes is provided. Diabetes can be type 1, type 2, pre-diabetes, gestational diabetes, or latent autoimmune diabetes of adults (LADA). In some cases, the diabetes is associated with a disorder that has caused damage to the pancreas, such as cystic fibrosis, chronic pancreatitis, or haemochromatosis.

In a further embodiment, a method of treating metabolic syndrome, a cluster of conditions such as high blood sugar and high triglycerides that can lead to cardiovascular disease, is provided.

In an additional embodiment, a method of smoking cessation and/or a method for preventing weight gain in former smokers is provided. In some embodiments, methods of treating cholestatic injury are provided.

The therapeutically effective dosage amount of any specific formulation will vary somewhat from drug to drug, patient to patient, and will depend upon factors such as the condition of the patient and the route of delivery. When administered conjointly with other pharmaceutically active agents, even less of the compounds of the disclosure may be therapeutically effective. Furthermore, the therapeutically effective amount may vary depending on the specific condition to be treated.

The compounds and compositions provided herein can be administered once or several times a day. The daily dose can be administered either by a single dose in the form of an individual dosage unit or several smaller dosage units or by multiple administration of subdivided dosages at certain intervals. Possible routes of delivery include buccally, subcutaneously, transdermally, intramuscularly, intravenously, orally, or by inhalation. Exemplary daily dosage ranges may be from about 0.1 mg to about 100 mg.

The compounds of the disclosure may be used with other types of therapy, including those which are non-drug based. Thus, in some embodiments, the methods of the present disclosure comprise administering to a subject a compound that that is capable of functioning as an antagonist of CB1 receptors in conjunction with one or more other types of non-drug-based therapy.

### Experimental Section

A number of compounds within the scope of Formula 1, above, were prepared and evaluated in various manners. Compounds were prepared generally according to Scheme 1, provided herein above.

Chemistry General. Purity and characterization of compounds were established by a combination of LC/MS, NMR, HPLC and TLC analytical techniques, as described below. 1H spectra were recorded on a Bruker Avance DPX-300 (300 MHz) spectrometer and were determined in chloroform-d (7.26 ppm) with TMS (0.00 ppm) as the internal reference. Chemical shifts are reported in ppm relative to the solvent signal and coupling constant (J) values are reported in hertz (Hz). TLC was performed with precoated silica gel 60 F254 plates, visualizing spots using UV light or I₂. LC/MS was performed with an Agilent InfinityLab MSD single quadrupole mass spectrometer equipped with an API-ES and an Agilent Infinity II 1260 HPLC equipped with an Agilent Infinity 1260 variable wavelength detector and a Phenomenex Synergi 2.5 μm Hydro-RP 100A C18 30×2 mm column. HPLC method for LC/MS: starting with a flow rate of 0.6 mL/min for 0.4 minutes at 20% solvent B followed by a 1.3 minute gradient of 20-95% solvent B at 0.6 mL/min followed by 2 minutes at 95% solvent B with a flow rate of 0.6 mL/min for 0.3 minutes and then a gradual ramp up of the flow rate to 1.2 mL/min at the end (solvent A, water with 0.1% formic acid; solvent B, acetonitrile with 0.1% formic acid and 5% water; absorbance monitored at 254 and 280 nm). MS method for LC/MS: using atmospheric pressure ionization-electrospray, positive and negative ions were monitored in the range of 70-700 or 200-2000. HPLC was performed with a Waters 2695 Separation Module equipped with a Waters 2996 Photodiode Array Detector and a Phenomenex Synergi 4 μm Hydro-RP 80A C18 250×4.6 mm column using a flow rate of 1 mL/min starting with 1 minute at 5% solvent B, followed by a 15 minute gradient of 5-95% solvent B, followed by 9 minutes at 95% solvent B (solvent A, water with 0.1% TFA; solvent B, acetonitrile with 0.1% TFA and 5% water; absorbance monitored at 220 and 280 nm).
Intermediate A: 6-Chloro-8-(2-chlorophenyl)-9-(4-chlorophenyl)-9H-purine. To a heterogeneous mixture of 5-amino-4,6-dichloropyrimidine (3.3 g, 20 mmol) and 4-chloroaniline (2.6 g, 1 equiv) in n-BuOH (40 mL) was added 6 N HCl (1.7 mL, 0.5 equiv). The mixture was heated at 100° C. for 15 h. At rt, ethyl acetate (40 mL) and 2 N NaOH (16 mL) were added and the mixture stirred vigorously for 15 min. Additional ethyl acetate (160 mL) and saturated NaHCO₃ solution (80 mL) were added. The aqueous layer was removed, and the organic layer washed with 0.8 M NaHCO₃ solution (50 mL). Celite (20 g) and toluene (20 mL) were added to the organic layer and most of the solvent evaporated. Purification by flash chromatography, using silica gel and an EtOAc/hexanes gradient, provided 5.0 g (98%) of the intermediate 5-amino-4-(4-chlorophenyl)-6-chloropyrimidine as a tan crystalline solid. R_{f}=0.17 (30% EtOAc/hexanes; UV active). LC/MS (m/z) 255.2 (M+1), 253.0 (M−1), >97% at 2.44 min. To a solution of 5-amino-4-(4-chlorophenyl)-6-chloropyrimidine (5.0 g, 19 mmol) and 2-chlorobenzaldehyde (4.5 mL, 2 equiv) in dioxane (60 mL) was added 15% FeCl₃/silica gel (11 g, 600 mg/mmol). The mixture was stirred at rt for 10 min and then at 95° C. for 20 h. At rt, the mixture was filtered using a sintered glass funnel (sand on sodium sulfate on celite with a medium frit) and washed with chloroform (60 mL). Toluene (6 mL) was added and most of the solvent evaporated. The resulting residue was dissolved in CH₂Cl₂ (60 mL) and cooled in an ice bath. DDQ (4.3 g, 1 equiv) was added and after 10 min, the ice bath was removed. The mixture was stirred at rt for 2 h and then most of the solvent was evaporated. Ethyl acetate (200 mL) was added and the resulting organic solution was washed with water (2×100 mL). Celite (20 g) was added, and the solvent was evaporated. Flash chromatography using silica gel with an EtOAc/hexanes gradient provided 5.2 g (73%) of a tan amorphous solid. R_{f}=0.37 (30% EtOAc/hexanes; blue w UV). LC/MS (m/z) 275.2 (M+1), >95% at 2.70 min. Note: to make 10 g of 15% FeCl₃/silica, add a solution of 1.5 g of FeCl₃ in 10 mL THF to 8.5 g of silica gel, stir for 1 h and then evaporate the solvent, thus providing a yellow tinted solid.

Intermediate B1 (R=H): 8-(2-Chlorophenyl)-9-(4-chlorophenyl)-6-(4-aminopiperidin-1-yl)-9H-purine. To a solution of A (1.3 g, 3.5 mmol) and 4-(N-Boc-amino) piperidine (0.83 g, 1.2 equiv) in NMP (7 mL) was added potassium carbonate (1.4 g, 3 equiv). The mixture was stirred at rt for 15 min and then at 80° C. for 15 h. Ethyl acetate (35 mL) was added, followed by brine (14 mL) and water (7 mL). The organic layer was washed with brine/water (3/1, 2×14 mL). Celite (6 g) was added to the organic layer and the solvent evaporated. Flash chromatography using silica gel with an EtOAc/hexanes gradient provided 1.9 g (100%) of the intermediate 8-(2-chlorophenyl)-9-(4-chlorophenyl)-6-(4-(N-Boc-amino) piperidin-1-yl)-9H-purine as an off white amorphous solid. R_{f}=0.37 (40% EtOAc/hexanes; blue w UV). LC/MS (m/z) 539.4 (M+1), >95% at 2.86 min. To a mixture of 8-(2-chlorophenyl)-9-(4-chlorophenyl)-6-(4-(N-Boc-amino) piperidin-1-yl)-9H-purine (1.9 g, 3.5 mmol) and ethanol (8 mL) was added 6 N HCl (4 mL). The mixture was stirred at rt for 10 min and then at 50° C. for 3 h. The mixture was cooled in an ice bath and chloroform (20 mL) was added, followed by brine (8 mL). 6 N NaOH (4.1 mL) was added slowly and after 5 min, the bath was removed. After 10 min, the layers were separated and the aqueous layer was saturated with NaCl. The aqueous layer was extracted with chloroform (2×10 mL). The combined organic layers were dried (Na₂SO₄ for 20 min), filtered and evaporated. Toluene (3 mL) was added, and the solvent was evaporated to provide 1.6 g (100%) of a tan amorphous solid. ¹H NMR (300 MHz, CDCl₃) δ 8.38 (s, 1H), 7.52 (d, J=6.6 Hz, 1H), 7.30-7.42 (m, 5H), 7.20 (d, J=8.7 Hz, 2H), 5.40 (bs, 2H), 3.27 (m, 2H), 2.92-3.11 (m, 1H), 1.89-2.10 (m, 2H), 1.31-1.51 (m, 4H). LC/MS (m/z) 439.4 (M+1), >95% at 2.22 min.

Intermediate B2 (R=Me): 8-(2-Chlorophenyl)-9-(4-chlorophenyl)-6-(4-amino-4-methylpiperidin-1-yl)-9H-purine. To a solution of A (752 mg, 2.0 mmol) and 4-(N-Boc-amino)-4-methylpiperidine (514 mg, 1.2 equiv) in NMP (6 mL) was added potassium carbonate (830 mg, 3 equiv). The mixture was stirred at rt for 15 min and then at 80° C. for 15 h. Ethyl acetate (20 mL) was added, followed by brine (12 mL) and water (4 mL). The organic layer was washed with brine/water (3/1, 2×6 mL). Celite (5 g) was added to the organic layer and the solvent evaporated. Flash chromatography using silica gel with an EtOAc/hexanes gradient provided 1.05 g (95%) of the intermediate 8-(2-chlorophenyl)-9-(4-chlorophenyl)-6-(4-(N-Boc-amino)-4-methylpiperidin-1-yl)-9H-purine as an off white amorphous solid. R_{f}=0.48 (40% EtOAc/hexanes; blue w UV). LC/MS (m/z) 553.2 (M+1), >97% at 3.28 min. To a mixture of 8-(2-chlorophenyl)-9-(4-chlorophenyl)-6-(4-(N-Boc-amino)-4-methylpiperidin-1-yl)-9H-purine (1.0 g, 1.8 mmol) and ethanol (4 mL) was added 6 N HCl (2 mL). The mixture was stirred at rt for 10 min and then at 50° C. for 15 h. At rt, ethyl acetate (15 mL) was added, followed by brine (4 mL) and then 6 N NaOH (2.2 mL). After 10 min, the aqueous layer was saturated with NaCl, and the layers were separated. The organic layer was dried (Na₂SO₄ for 20 min) and filtered. Toluene (2 mL) was added, and the solvent evaporated to provide 837 mg (100%) of a tan amorphous solid. ¹H NMR (300 MHz, CDCl₃) δ 8.36 (s, 1H), 7.50 (dd, J=7.1, 1.0 Hz, 1H), 7.29-7.40 (m, 5H), 7.18 (d, J=7.1 Hz, 2H), 4.55 (br s, 2H), 4.20 (br s, 2H), 1.71 (ddd, J=13.3, 9.1, 3.9 Hz, 2H), 1.53-1.64 (m, 2H), 1.22 (s, 3H). LC/MS (m/z) 453.0 (M+1), >95% at 2.66 min.
N-{1-[8-(2-Chlorophenyl)-9-(4-chlorophenyl)-9H-purin-6-yl]piperidin-4-yl}-2-(2-fluorophenyl)acetamide (1): To a solution of 2-flourobenzylcarboxylic acid (19 mg, 1.2 equiv) and BOP (53 mg, 1.2 equiv) in MeCN (1 mL) was added NEt₃ (0.031 mL, 2.2 equiv). The mixture was stirred at rt for 30 minutes and then intermediate B1 (44 mg, 0.1 mmol) was added. After an additional 30 minutes at rt, the mixture was heated at 50° C. for 15 h (heterogeneous). At rt, ethyl acetate (4 mL) was added, followed by brine (0.8 mL) and water (0.4 mL). After 10 minutes, the aqueous layer was removed, and the organic layer was washed with 0.5 M NaHCO₃ solution (0.6 mL). Celite (600 mg) was added to the organic layer and the solvent evaporated. Flash chromatography using silica gel, eluting with an EtOAc/hexanes gradient containing 10% CH₂Cl₂ and up to 2% MeOH provided 56 mg (97%) of a white amorphous solid, mp 204-205° C. R_{f}=0.30 (2% MeOH/60% EtOAc/hexanes; blue with UV). ¹H NMR (300 MHz, CDCl₃) δ 8.36 (s, 1H), 7.49 (d, J=6.6 Hz, 1H), 7.29-7.43 (m, 7H), 7.18 (d, J=8.5 Hz, 2H), 6.99-7.14 (m, 2H), 5.22-5.54 (m, 3H), 4.01-4.25 (m, 1H), 3.58 (s, 2H), 3.25-3.39 (m, 2H), 2.02-2.14 (m, 2H), 1.36-1.52 (m, 2H). LC/MS (m/z) 575.0 (M+1), >98% at 2.90 min. HPLC>99% at 15.84 min.
N-{1-[8-(2-Chlorophenyl)-9-(4-chlorophenyl)-9H-purin-6-yl]piperidin-4-yl}-2-(4-fluorophenyl)acetamide (2): To a solution of 4-flourobenzylcarboxylic acid (19 mg, 1.2 equiv) and BOP (53 mg, 1.2 equiv) in MeCN (1 mL) was added NEt₃ (0.031 mL, 2.2 equiv). The mixture was stirred at rt for 30 minutes and then intermediate B1 (44 mg, 0.1 mmol) was added. After an additional 30 minutes at rt, the mixture was heated at 50° C. for 15 h (heterogeneous). At rt, ethyl acetate (4 mL) was added, followed by brine (0.8 mL) and water (0.4 mL). After 10 minutes, the aqueous layer was removed, and the organic layer was washed with 0.5 M NaHCO₃ solution (0.6 mL). Celite (600 mg) was added to the organic layer and the solvent evaporated. Flash chromatography using silica gel, eluting with an EtOAc/hexanes gradient containing 10% CH₂Cl₂ and up to 2% MeOH provided 46 mg (80%) of a white amorphous solid, mp 202-203° C. R_{f}=0.22 (2% MeOH/60% EtOAc/hexanes; blue with UV). ¹H NMR (300 MHz, CDCl₃) δ 8.36 (s, 1H), 7.49 (d, J=6.8 Hz, 1H), 7.28-7.43 (m, 5H), 7.13-7.25 (m, 4H), 6.94-7.08 (m, 2H), 5.19-5.51 (m, 3H), 4.01-4.26 (m, 1H), 3.53 (s, 2H), 3.23-3.37 (m, 2H), 2.01-2.13 (m, 2H), 1.32-1.51 (m, 2H). LC/MS (m/z) 575.0 (M+1), 619.0 (M−1+46), >98% at 2.90 min. HPLC>99% at 15.94 min.
N-{1-[8-(2-Chlorophenyl)-9-(4-chlorophenyl)-9H-purin-6-yl]piperidin-4-yl}-2-(2,4-difluorophenyl)acetamide (3): To a solution of 2,4-diflourobenzylcarboxylic acid (21 mg, 1.2 equiv) and BOP (53 mg, 1.2 equiv) in MeCN (1 mL) was added NEt₃ (0.031 mL, 2.2 equiv). The mixture was stirred at rt for 30 minutes and then intermediate B1 (44 mg, 0.1 mmol) was added. After an additional 30 minutes at rt, the mixture was heated at 50° C. for 15 h (heterogeneous). At rt, ethyl acetate (4 mL) was added, followed by brine (0.8 mL) and water (0.4 mL). After 10 minutes, the aqueous layer was removed, and the organic layer was washed with 0.5 M NaHCO₃ solution (0.6 mL). Celite (600 mg) was added to the organic layer and the solvent evaporated. Flash chromatography using silica gel, eluting with an EtOAc/hexanes gradient containing 10% CH₂Cl₂ and up to 2% MeOH provided 59 mg (100%) of a white amorphous solid, mp 152-154° C. R_{f}=0.37 (2% MeOH/60% EtOAc/hexanes; blue with UV). ¹H NMR (300 MHz, CDCl₃, CD₃OD) δ 8.33 (s, 1H), 7.50 (d, J=7.0 Hz, 1H), 7.27-7.44 (m, 6H), 7.19 (d, J=8.5 Hz, 2H), 6.73-6.95 (m, 2H), 6.22 (d, J=6.6 Hz, 1H), 5.30-5.41 (m, 2H), 4.05-4.16 (m, 1H), 3.52 (s, 2H), 3.22-3.45 (m, 2H), 2.05-2.16 (m, 2H), 1.40-1.61 (m, 2H). LC/MS (m/z) 593.2 (M+1), 637.2 (M−1+46), >98% at 3.08 min. HPLC>99% at 16.25 min.
N-{1-[8-(2-Chlorophenyl)-9-(4-chlorophenyl)-9H-purin-6-yl]-4-methylpiperidin-4-yl}-2-(2-fluorophenyl)acetamide (4): To a solution of 2-flourobenzylcarboxylic acid (19 mg, 1.2 equiv) and HBTU (46 mg, 1.2 equiv) in MeCN (1 mL) was added NEt₃ (0.031 mL, 2.2 equiv). The mixture was stirred at rt for 30 minutes and then intermediate B2 (45 mg, 0.1 mmol) was added. After an additional 30 minutes at rt, the mixture was heated at 50° C. for 15 h (heterogeneous). At rt, ethyl acetate (4 mL) was added, followed by brine (0.8 mL) and water (0.4 mL). After 10 minutes, the aqueous layer was removed, and the organic layer was washed with 0.5 M NaHCO₃ solution (0.6 mL). Celite (600 mg) was added to the organic layer and the solvent evaporated. Flash chromatography using silica gel, eluting with an EtOAc/hexanes gradient containing 10% CH₂Cl₂ and up to 2% MeOH provided 52 mg (88%) of a white amorphous solid, mp 105-106° C. R_{f}=0.41 (60% EtOAc/hexanes; blue with UV). ¹H NMR (300 MHz, CDCl₃) δ 8.36 (s, 1H), 7.49 (d, J=6.6 Hz, 1H), 7.29-7.42 (m, 7H), 7.04-7.22 (m, 4H), 5.33 (s, 1H), 4.80 (br s; suppressed), 3.58 (s, 2H), 3.55 (br s, 2H), 2.12-2.23 (m, 2H), 1.65-1.75 (m, 2H), 1.45 (s, 3H). LC/MS (m/z) 589.2 (M+1), 633.2 (M−1+46), >97% at 3.16 minutes. HPLC 99% at 16.47 minutes.
N-{1-[8-(2-Chlorophenyl)-9-(4-chlorophenyl)-9H-purin-6-yl]-4-methylpiperidin-4-yl}-2-(4-fluorophenyl)acetamide (5): To a solution of 4-flourobenzylcarboxylic acid (19 mg, 1.2 equiv) and HBTU (46 mg, 1.2 equiv) in MeCN (1 mL) was added NEt₃ (0.031 mL, 2.2 equiv). The mixture was stirred at rt for 30 minutes and then intermediate B2 (45 mg, 0.1 mmol) was added. After an additional 30 minutes at rt, the mixture was heated at 50° C. for 15 h (heterogeneous). At rt, ethyl acetate (4 mL) was added, followed by brine (0.8 mL) and water (0.4 mL). After 10 minutes, the aqueous layer was removed, and the organic layer was washed with 0.5 M NaHCO₃ solution (0.6 mL). Celite (600 mg) was added to the organic layer and the solvent evaporated. Flash chromatography using silica gel, eluting with an EtOAc/hexanes gradient containing 10% CH₂Cl₂ and up to 2% MeOH provided 55 mg (93%) of a white amorphous solid, mp 95-96° C. R_{f}=0.27 (60% EtOAc/hexanes; blue with UV). ¹H NMR (300 MHz, CDCl₃) δ 8.37 (s, 1H), 7.49 (d, J=6.8 Hz, 1H), 7.29-7.42 (m, 5H), 7.14-7.25 (m, 4H), 7.05 (t, J=8.5 Hz, 2H), 5.18 (s, 1H), 4.84 (br s; suppressed), 3.53 (br s, 4H), 2.09-2.20 (m, 2H), 1.67-1.79 (m, 2H), 1.45 (s, 3H). LC/MS (m/z) 589.2 (M+1), 633.2 (M−1+46), >98% at 3.15 minutes. HPLC 98% at 16.60 minutes.
Relevant in vitro data for various compounds referenced above (and falling within the scope of Formula 1 provided herein), is presented below in Table 1 below and discussed further therein below.

TABLE 1
Compounds and In Vitro Study Results

| | | | Ke | Ki | Selectivity | |
|---|---|---|---|---|---|---|
| | | | hCB1 | hCB1 | Ki | |
| Compound | R | R′ | (nm) | (nm)^{a} | (CB2/CB1) | HLM |
| 1 | H | 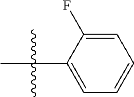 [SMILES: *c1ccccc1F] | 1.0 | 1.5 | 110 | HL: 146 min Cl: 9 μL/min/mg |
| 2 | H | 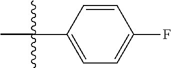 [SMILES: *c1ccc(F)cc1] | 1.0 | 1.4 | 210 | HL: 150 min Cl: 8 μL/min/mg |
| 3 | H | 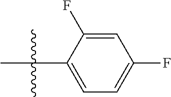 [SMILES: *c1ccc(F)cc1F] | 0.8 | 1.1 | 510 | HL: 133 min Cl: 9 μL/min/mg |
| 4 | Me | 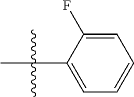 [SMILES: *c1ccccc1F] | 3.8 | | | HL: ND Cl: ND |
| 5 | Me | 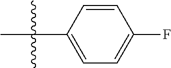 [SMILES: *c1ccc(F)cc1] | 1.8 | | | HL: ND Cl: ND |

^{a}Displacement was measured using [³H]CP55940 in CHO cell membrane preparations overexpressing
human CB1 or human CB2 receptors

As demonstrated in Table 1, the prepared compounds exhibited excellent binding activity. See Ki values above, based on radioligand displacement assay using ^{3H}-CP55940, a synthetic cannabinoid, (−)-cis-3-[2-hydroxy-4-(1,1-dimethylheptyl)phenyl]-trans-4-(3-hydroxypropyl)cyclohexanol, which is a compound commonly used to study the endocannabinoid system.

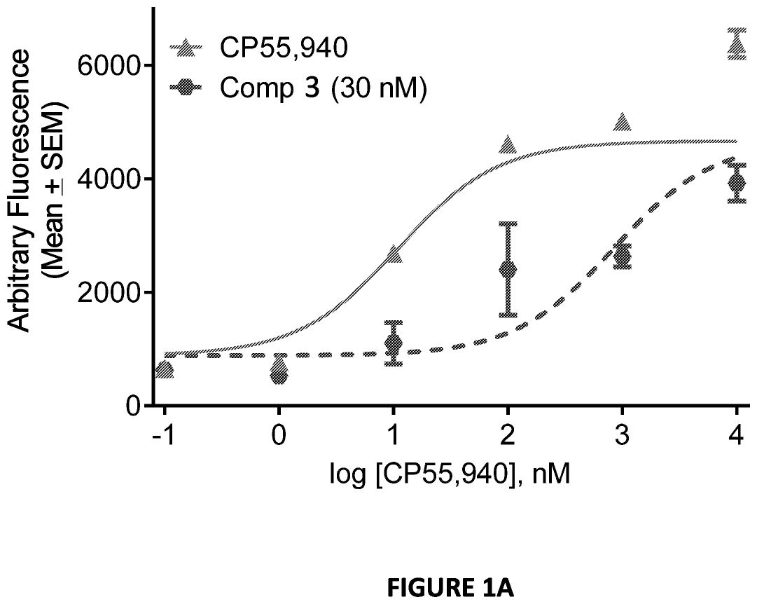

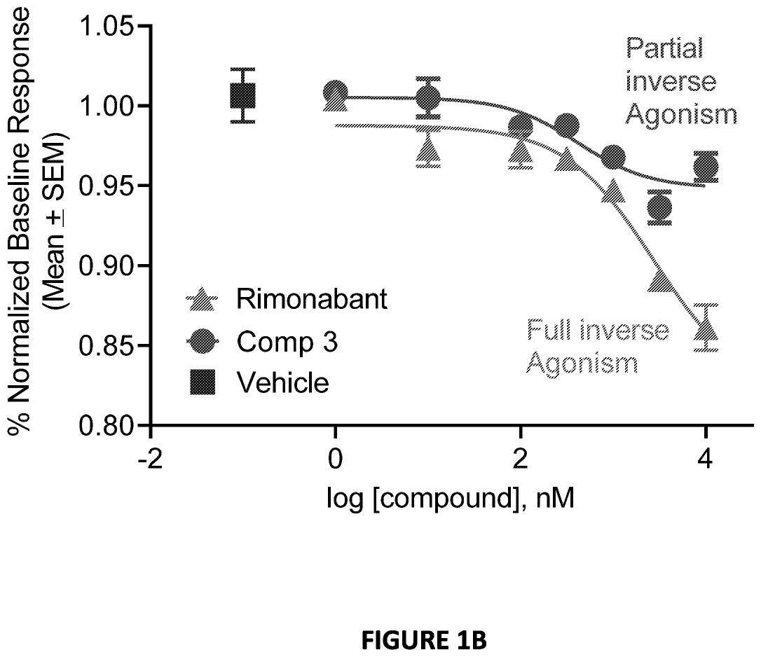

FIGS. 1A and 1B provide results of a high-resolution calcium mobilization assay that demonstrates the activity of tested compounds. A functional fluorescent calcium mobilization was performed in CHO cells expressing human CB1 along with G_{qα16}. In FIG. 1A, a concentration-dependent increase in fluorescence upon treatment with the CB1 agonist CP55,940 is shown (solid line/triangle markers). Pre-incubation with Compound 3 according to the present disclosure (see Table 1) leads to antagonism as indicated by a rightward shift of the CP55,940 curve (dotted line/hexagon markers). The EC₅₀ values in the presence and absence of the antagonist are used to calculate antagonist apparent equilibrium dissociation constant (Ke). Suppression of basal signaling in the absence of any stimulation/agonism was measured using the same approach. The same fluorescent calcium mobilization assay was used to measure the effect of the prototypical CB1 receptor full inverse agonist rimonabant (triangle markers) along with the newly discovered Compound 3 (circle markers) (FIG. 1B). Note partial suppression of maximal response (~30% of rimonabant′ Emax) with this compound versus with rimonabant. Rimonabant suppresses basal signaling (inverse agonism) but Compound 3 behaves as a partial inverse agonist. Provided data are representative of a minimum of three experiments and were fitted using non-linear regression analyses in GraphPad Prism.

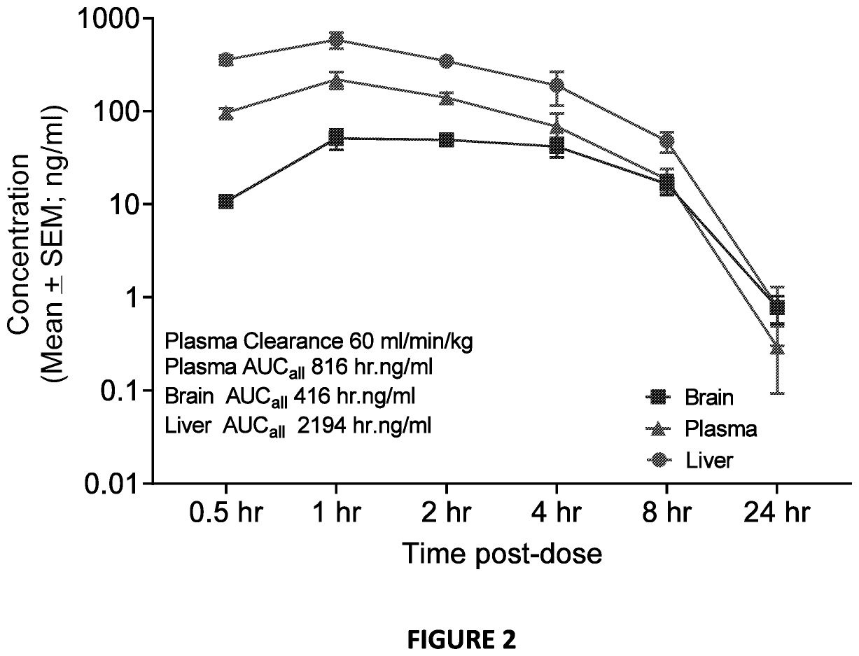

FIG. 2 provides results of a study that demonstrates brain penetration of Compound 3. C57BL6 female mice (n=3) were orally dosed with Compound 3 at 2.5 mpk in a 2% NMP/canola oil vehicle. Animals were sacrificed at various time points and tissue samples were collected. Whole body perfusion was conducted prior to collection of brain samples to remove residual blood. Samples were analyzed using LC-MS and WinNonLin software was used for data analysis. AUC=Area under Curve.

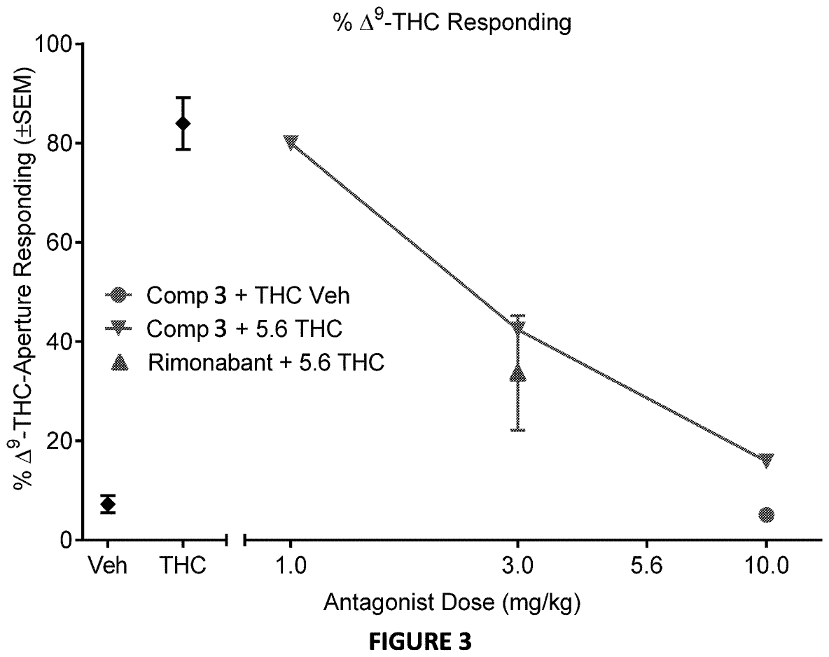

As shown in FIG. 3, Compound 3 was further analyzed to evaluate antagonism in a THC drug discrimination model. Drug discrimination is a behavioral model of the interoceptive effects of a compound. This pharmacologically selective model has high predictive validity, producing a strong correlation between drugs that have sharing discriminative stimulus properties in animals and drugs that humans identify as having similar subjective effects. FIG. 3 provides the results of this study, in which C57BL6 male mice (n=8) were trained to discriminate the reference drug (THC) using a discrimination procedure similar to that described in Wiley et al., Neuropharmacology 75, 145-154 (2013), Van et al., Eur. J. Pharmacol. 615, 102-107, and Wiley et al., J. Pharmacol. Exp. Ther. 354, 328-339 (2015), which are incorporated herein by reference in their entireties. For each session, the percentage of responses on the drug-associated lever and response rate response(s) were calculated. Compound 3 was found to antagonize THC drug discrimination in the mice; maximum inhibition was comparable to that of rimonabant. This data is considered to be highly significant when compared to a 5.6 mpk THC training dose+vehicle (left x axis) using ANOVA (p<0.0001) at 3, 5.6, and 10 mpk dose of each antagonist. Response rates were not significantly altered compared to controls (not shown). These significant results confirm the in vivo activities of Compound 3 in a highly relevant model of human THC abuse.

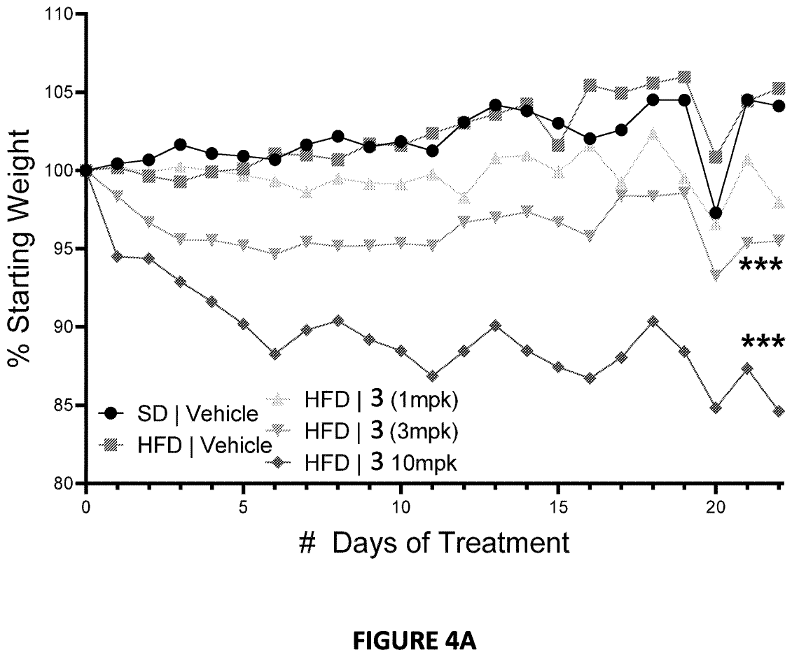

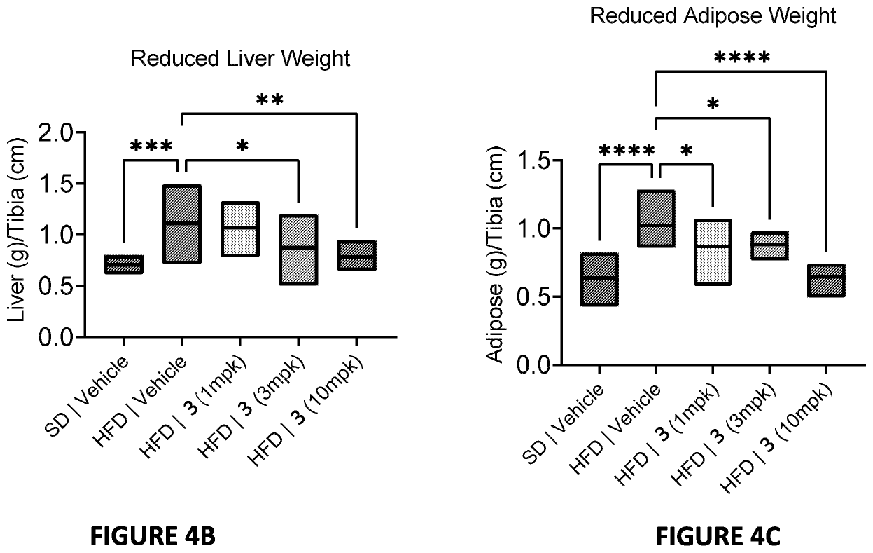

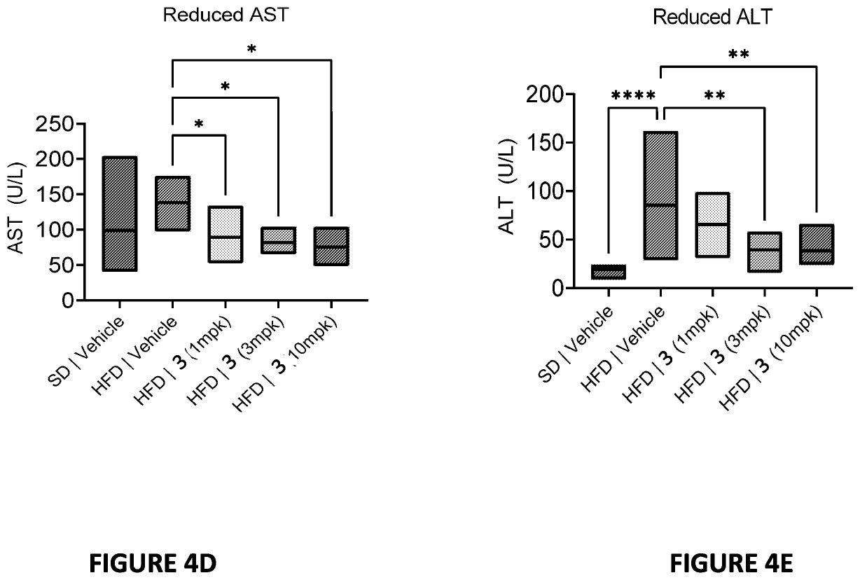

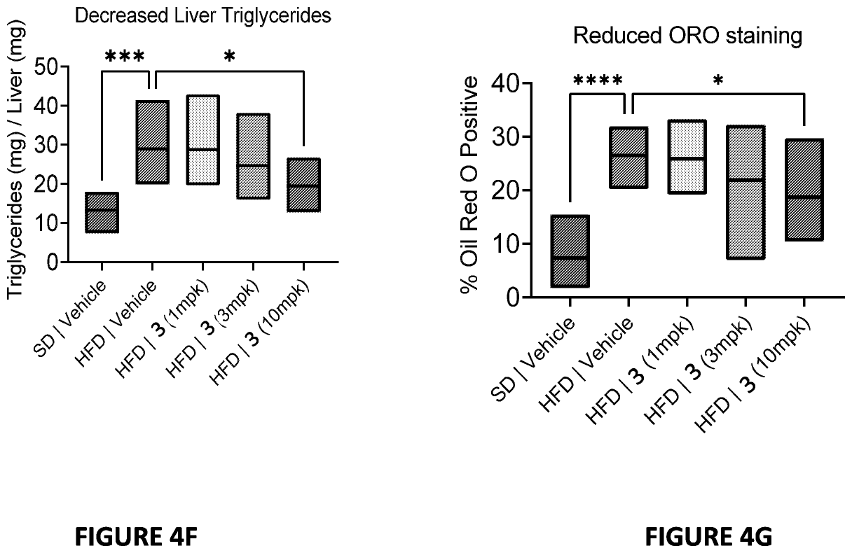

FIGS. 4A-4J demonstrate reversal of diet-induced obesity (DIO) in mice with Compound 3. Preconditioned male C57BL/6J mice were purchased from the Jackson Laboratories (Bar Harbor, Maine) at 18 weeks of age. These animals were maintained either on ad libitum standard diet (SD) with 10% fat (D12450B) or on 60% high fat diet (HFD; D12492) from Research Diets (New Brunswick, NJ.) Animals were dosed by intragastric gavage once daily Monday-Friday (5 days per week) with either vehicle (0.5% sodium carboxymethylcellulose with 1% NMP and 0.3% Tween 80 in de-ionized water) or Compound 3 suspended in the vehicle. Body weights were recorded at regular intervals. Organs were removed and processed as has been described in the legend of FIG. 4. Biomarkers of efficacy were measured including circulating enzymes, liver weight and liver adiposity as indicated.

As shown, treatment with Compound 3 (oral, 1× daily, 5-days per week) led to significantly reduced body weight (FIG. 4A), liver weight (FIG. 4B), and epididymal adipose weight (FIG. 4C) normalized to tibia length. Data were reported as mean (FIG. 4A) or mean and range (FIGS. 4B and 4C). Treatment with Compound 3 was found to reduce circulating transaminases AST (FIG. 4D) and ALT (FIG. 4E) and to decrease hepatic cholesterol content (FIG. 4F). Hepatic statosis was reduced, as assessed using Oil Red O staining (FIG. 4G), with representative images shown as FIGS. 4H, 4I, and 4J. Significance was calculated using ANOVA with Fisher's test (p<0.05*, 0.01**, 0.001***, 0.0001****).

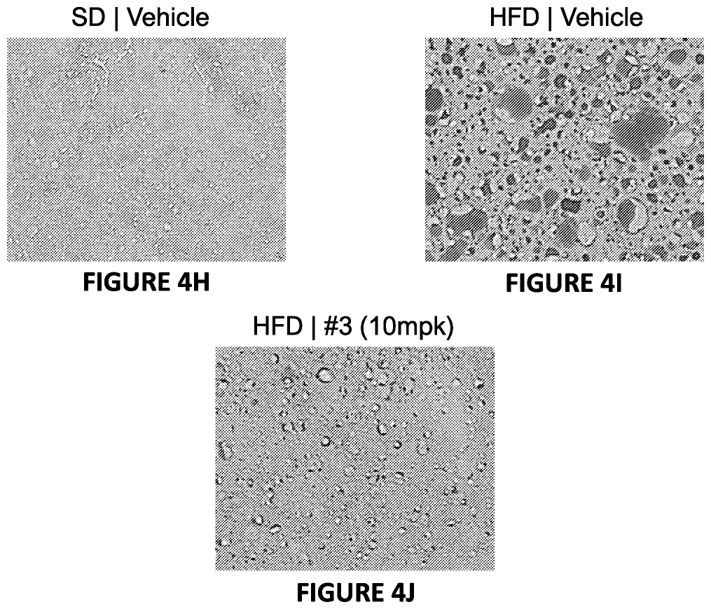

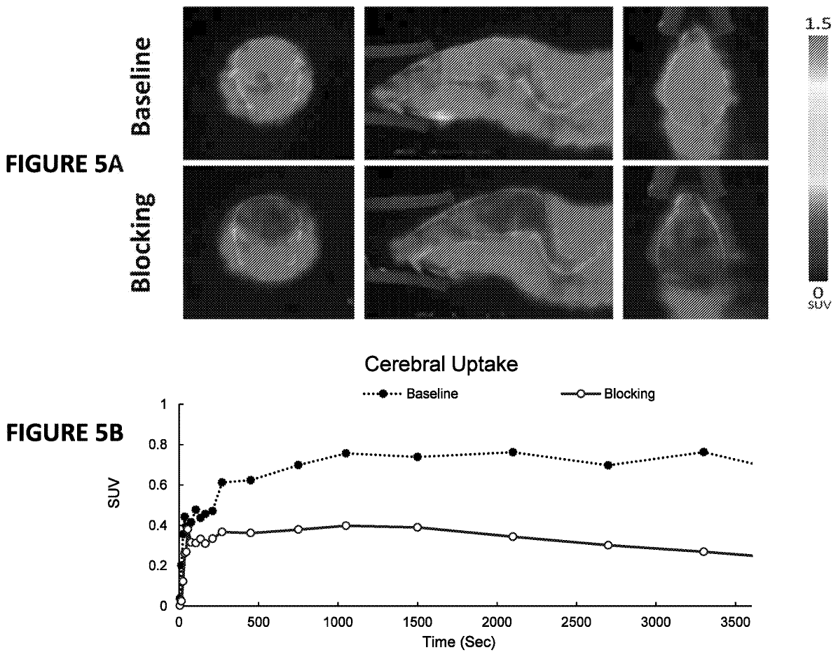

FIG. 5A provides a representative image of target specificity of Compound 3 for the CB1 receptor in mice along with quantification. Positron emission tomography (PET) studies were used to assess brain penetration and target engagement of Compound 3 in both male and female B6 mice. The well-characterized CB1 radiotracer ¹¹C-JHU75528 was used for this purpose. A baseline response for each animal (2 male and 2 female mice) was first established using the vehicle and tracer (~10 MBq in 0.1-0.15 ml volume, tail vein catheter). Following a 48-hr washout, animals were administered Compound 3 via intraperitoneal (IP) injection followed by the probe. PET image acquisition by dynamic scanning was performed for 65 min after the second injection with energy window of 350-700 KeV. See, e.g., FIG. 5A, where the top series provides baseline images, and the bottom series shows a blockade of the signal upon pretreatment with Compound 3. The PET images were normalized to the standardized uptake value (SUV) unit. Two-tissue compartmental model was applied using the PKIN module in PMOD 4.0 software. See, e.g., FIG. 5B, where quantification of the signal confirmed a blocking effect on cerebral uptake. Compound 3 blocked ¹¹C-JHU75528 uptake, confirming in vivo activity.

Many modifications and other embodiments of the disclosed compounds, compositions, and methods set forth herein will come to mind to one skilled in the art to which this disclosure pertains having the benefit of the teachings presented in the foregoing descriptions and the associated drawings. Therefore, it is to be understood that the disclosure is not to be limited to the specific embodiments described herein and that modifications and other embodiments are intended to be included within the scope of the appended claims. Although specific terms are employed herein, they are used in a generic and descriptive sense only and not for purposes of limitation.

1. A compound according to the structure: 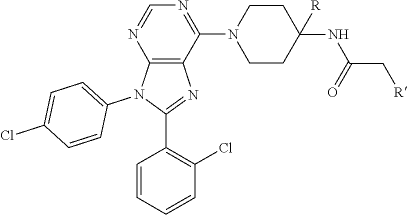
wherein:
R is H or methyl;
R′ is aryl or heteroaryl, optionally substituted with one or two substituents independently selected from the group consisting of F, Cl, methyl, CF₃, OCF₃, and OCHF₂,
or a pharmaceutically acceptable ester, amide, salt, solvate, prodrug, or isomer thereof.

2. The compound of claim 1, wherein R is H.

3. The compound of claim 1, wherein R is methyl.

4. The compound of claim 1, wherein R′ is aryl, optionally substituted with one or two substituents.

5. The compound of claim 1, wherein R′ is heteroaryl, optionally substituted with one or two substituents.

6. The compound of claim 1, wherein R′ is substituted with one or two substituents.

7. The compound of claim 1, wherein R′ is phenyl substituted with one or two F substituents.

8. The compound of claim 1, wherein R′ is selected from the group consisting of:

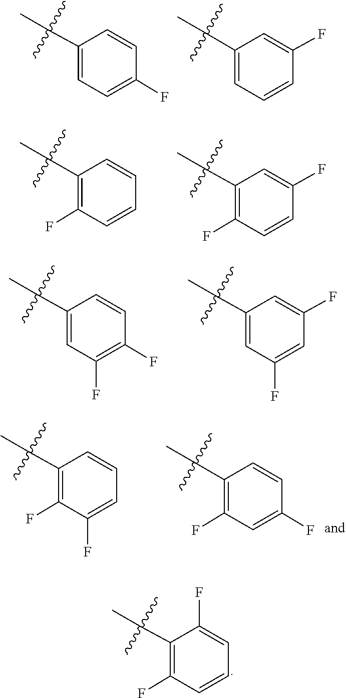

9. The compound of claim 1, in the form of a pharmaceutically acceptable salt.

10. A pharmaceutical composition, comprising the compound of claim 1 and one or more pharmaceutically acceptable carriers.

11. The pharmaceutical composition of claim 10, further comprising one or more additional active agents.

12. A method for treating or delaying the progression of disorders that are alleviated by antagonism of the CB1 receptor, the method comprising administering the compound of claim 1.

13. The method of claim 12, wherein the disorder is selected from the group consisting of metabolic syndrome, binge eating disorder, Preder Willi syndrome (PWS), and substance use disorders.

14. The method of claim 13, wherein the substance use disorder is selected from the group consisting of addiction to stimulants, depressants, or cannabis, alcohol consumption, nicotine consumption, acute cannabinoid-induced toxicity, and cannabinoid hyperemesis syndrome.

15. A method for treating or delaying the progression of disorders that are alleviated by antagonism of the CB1 receptor, the method comprising administering the pharmaceutical composition of claim 10.

16. The method of claim 15, wherein the disorder is selected from the group consisting of metabolic syndrome, binge eating disorder, Preder Willi syndrome (PWS), and substance use disorders.

17. The method of claim 16, wherein the substance use disorder is selected from the group consisting of addiction to stimulants, depressants, or cannabis, alcohol consumption, nicotine consumption, acute cannabinoid-induced toxicity, and cannabinoid hyperemesis syndrome.

18. The compound of claim 1, wherein R′ is

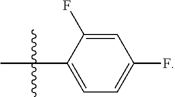 [SMILES: *c1ccc(F)cc1F]

19. The compound of claim 1, wherein R is H and wherein R′ is selected from the group consisting of:

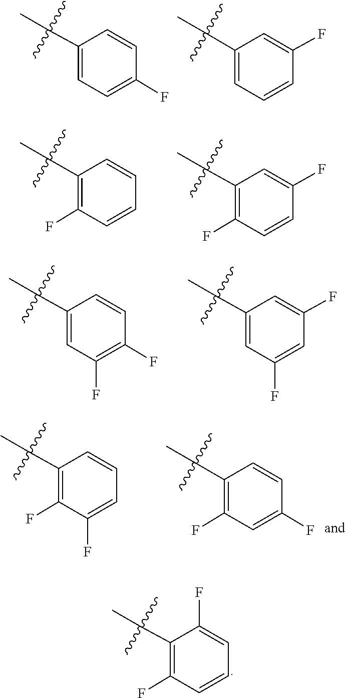

20. The compound of claim 1, wherein R is H and R′ is 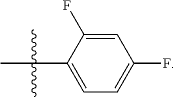 [SMILES: *c1ccc(F)cc1F]
